(19) United States
(12) Patent Application Publication
(10) Pub. No.: US 2019/0043616 A1
(43) Pub. Date: Feb. 7, 2019

(54) SYSTEMS AND METHODS FOR PERSONAL EMERGENCY

(71) Applicant: iBeat, Inc., San Francisco, CA (US)

(72) Inventors: Ryan Howard, San Francisco, CA (US); Brian Boarini, San Francisco, CA (US); Lawrence Stone, San Francisco, CA (US); Christopher Bumgardner, San Francisco, CA (US); Steven Szabados, Sausalito, CA (US)

(21) Appl. No.: 15/981,144

(22) Filed: May 16, 2018

Related U.S. Application Data
(60) Provisional application No. 62/541,029, filed on Aug. 3, 2017.

(51) Int. Cl. G16H 50/20; G06F 1/16

(52) U.S. Cl. CPC G16H 50/20; G06F 1/163

(57) ABSTRACT

In various embodiments, a personal emergency detection, notification, coordination and response method and system for individuals is disclosed. A monitoring device, for example a digital smart watch, is configured to identify an emergency using built-in sensors. The sensors are operable to perform spectral analysis of skin tissues. Example sensors include: LEDs and optical detectors for heart rate monitoring, blood perfusion checking, and tissue oxygenation checking; acceleration sensing to sense falls and accidents; and a GPS system for reporting the location of the wearer to interested parties. A communication chip with an associated antenna, and an audio chip are also included. Deviations in vital signs are used to detect health anomalies. By aggregating data that is anonymously collected from multiple users, the system constructs models that are compared with data from a specific user, to warn the user before an emergency occurs, for certain classes of health incidents.

### CROSS-REFERENCE TO RELATED APPLICATION

This application claims the benefit of U.S. Provisional Patent Application No. 62/541,029 filed Aug. 3, 2017, entitled “A Personal Emergency System for Emergency Identification, Emergency Notification, and Emergency Response for an Individual,” by Ryan HOWARD et al., which is hereby incorporated by reference.

### BACKGROUND

Health monitoring devices have become available in a wearable format, such as worn on a user's wrist. Many of them have the capability to monitor heart rate but are limited with respect to system intelligence. There is a need in the art for improved devices as well as improved methods for interfacing with them, to enable more sophisticated analysis of vital signs, more effective engagement of available resources, and overall more timely assistance to users undergoing health emergencies. There is a further need for a system architecture that can aggregate data from multiple users anonymously, perform analysis on the data collected, compare user data with behavioral models or standards determined from the analysis, and predict a health emergency before it would otherwise occur.

### SUMMARY

Various embodiments in accordance with the present disclosure can relate to the field of wearable health sensors, and more particularly to intelligent systems comprising wearable health sensors.

In various embodiments, a personal emergency detection, notification, coordination and response method and system for individuals is disclosed. A monitoring device, for example a digital smart watch, is configured to identify an emergency using built-in sensors. The sensors are operable to perform spectral analysis of skin tissues. Example sensors include: LEDs and optical detectors for heart rate monitoring, blood perfusion checking, and tissue oxygenation checking; acceleration sensing to sense falls and accidents; and a GPS system for reporting the location of the wearer to interested parties. A communication chip with an associated antenna, and an audio chip are also included. Deviations in vital signs are used to detect health anomalies. By aggregating data that is anonymously collected from multiple users, the system constructs models that are compared with data from a specific user, to warn the user before an emergency occurs, for certain classes of health incidents.

In various embodiments, a wearable health sensing system includes an enclosure and a clasp or band for attaching the enclosure to a user's body. In addition, the wearable health sensing system includes a processor, a memory containing instructions to be executed by the processor, a communication chip, an antenna associated with the communication chip, a Global Positioning Satellite (GPS) chip, a skin tissue sensor, and an energy storage device. Furthermore, the wearable health sensing system includes a charging interface for replenishing or recharging the energy storage device. Note that the processor is operable to process data sensed by the skin tissue sensor to detect anomalies that may occur in the user's vital signs, decide on an appropriate response, notify a support system, and coordinate emergency care of the user. The health sensing system is further configurable to aggregate data from multiple users anonymously, and applies additional analysis to establish norms of behavior, and by comparing user data against the norms of behavior, predict some user health emergencies before they would otherwise occur.

In various embodiments, the skin tissue sensor of the previous paragraph includes an array of photo diodes and detectors. In various embodiments, the skin tissue sensor is operable to perform spectral analysis. In various embodiments, the wearable health sensing system of the previous paragraph further includes an accelerometer for sensing activity level and physical mishaps. In various embodiments, the wearable health sensing system further includes a speaker for producing sound, for signaling the user or nearby persons. In various embodiments, the wearable health sensing system further includes an interactive display. In various embodiments, the wearable health sensing system further includes a capacitive sensor for the health sensing system to determine if good contact is made between the skin tissue sensor and the user's skin. In various embodiments, the additional analysis of the previous paragraph can include machine learning. In various embodiments, the wearable health sensing system further includes a diversity antenna to enhance the performance of the antenna.

In various embodiments, a method involves identifying a medical emergency of a user, notifying support systems, and coordinating emergency care. The method includes providing a digital monitoring device in the form of a wearable device; providing a processor and memory for operating the digital monitoring device; providing a plurality of spaced apart light emitting diodes (LEDs) having multiple frequencies; and positioning the spaced apart LEDs adjacent the user's skin. Furthermore, the method includes measuring reflections from the user's skin; recording the measured reflections as absorption spectra; analyzing the absorption spectra to determine the vital signs; and detecting an abnormality. Moreover, the method includes deciding on an appropriate response to the abnormality; notifying one or more support systems if an abnormality has occurred; and coordinating emergency care to be administered to the user. In addition, the method includes aggregating data from multiple users to anonymously create standards for normal and abnormal data sets; and comparing the user's data set against the normal and abnormal data sets to provide early warning to the user of a potential health emergency.

In various embodiments, the method of the previous paragraph includes signaling the user via a vibrating element in the digital monitoring device. In various embodiments, the method includes signaling of the user via tone or voice. In various embodiments, the method includes signaling of nearby persons via tone or voice. In various embodiments, the method includes providing confirmation by the user that an incident has occurred. In various embodiments, the method includes assuming confirmation that an incident has occurred if the user is incapacitated. In various embodiments, the method includes coordinating emergency responses through a dispatch center. In various embodiments, the method includes engaging additional responders who are in the same location as the user. In various embodiments, the method includes providing acceleration sensing and using this data to notify interested parties of falls or accidents. In various embodiments, the method includes notifying interested parties of health incidents detected by the digital monitoring device. In various embodiments, the method includes coordinating individuals and systems based on data to expedite emergency care. In various embodiments, the method includes providing tissue oxygenation sensing and reporting. In various embodiments, the method includes providing pulse rate sensing and reporting. In various embodiments, the method includes determining if the wearable device is properly positioned adjacent the user's skin. In various embodiments, the method includes providing ambient temperature sensing and using this information to calibrate the digital monitoring device. In various embodiments, the LED measurements of the previous paragraph utilize spatially resolved spectroscopy.

In various embodiments, a health sensing system includes a processor and memory for operating the health sensing system; a plurality of spaced apart LEDs in contact with the user's body and having multiple frequencies; a plurality of spaced apart photo detectors for measuring light sourced from the spaced apart LEDs that subsequently diffuses through the user's body; an array of conductive components in contact with the user's body; an array of sensing circuits associated with the array of conductive components for determining correct disposition of the health sensing system relative to the user's body; absorption spectra determined from the measurements of light sourced from the spaced apart LEDs that subsequently diffuses through the user's body; and user's vital signs determined from the absorption spectra. Note that the processor is operable to make inferences about the health of the user determined from the vital signs. In addition, the processor is operable to aggregate data from multiple users to anonymously create standards for normal and abnormal data sets. Moreover, the processor is operable to compare the user's vital signs against the normal and abnormal data sets to provide early warning to the user of a potential health emergency.

While various embodiments in accordance with the present disclosure have been specifically described within this Summary, it is noted that the claimed subject matter are not limited in any way by these various embodiments.

### BRIEF DESCRIPTION OF THE DRAWINGS

Within the accompanying drawings, various embodiments in accordance with the present disclosure are illustrated by way of example and not by way of limitation. It is noted that like reference numerals denote similar elements throughout the drawings.

FIG. 1 shows a cross-sectional view of a wrist worn health monitoring device in accordance with various embodiments of the present disclosure.

FIG. 2 is a flow diagram that illustrates an exemplary computer implemented method for providing timely emergency assistance to a user whose vital signs have deviated from the normal range in accordance with various embodiments of the present disclosure.

FIG. 3 is a block diagram of an example of a computing system upon which one or more various embodiments described herein may be implemented in accordance with various embodiments of the present disclosure.

### DETAILED DESCRIPTION

Reference will now be made in detail to various embodiments in accordance with the present disclosure, examples of which are illustrated in the accompanying drawings. While described in conjunction with various embodiments, it will be understood that these various embodiments are not intended to limit the present disclosure. On the contrary, the present disclosure is intended to cover alternatives, modifications and equivalents, which may be included within the scope of the present disclosure as construed according to the Claims. Furthermore, in the following detailed description of various embodiments in accordance with the present disclosure, numerous specific details are set forth in order to provide a thorough understanding of the present disclosure. However, it will be evident to one of ordinary skill in the art that the present disclosure may be practiced without these specific details or with equivalents thereof. In other instances, well known methods, procedures, components, and circuits have not been described in detail so as not to unnecessarily obscure aspects of the present disclosure.

Some portions of the detailed descriptions that follow are presented in terms of procedures, logic blocks, processing, and other symbolic representations of operations on data bits within a computer memory. These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those utilizing physical manipulations of physical quantities. Usually, although not necessarily, these quantities take the form of electrical or magnetic signals capable of being stored, transferred, combined, compared, and otherwise manipulated in a computing system. It has proven convenient at times, principally for reasons of common usage, to refer to these signals as transactions, bits, values, elements, symbols, characters, samples, pixels, or the like.

It should be borne in mind, however, that all of these and similar terms are to be associated with the appropriate physical quantities and are merely convenient labels applied to these quantities. Unless specifically stated otherwise as apparent from the following discussions, it is appreciated that throughout the present disclosure, discussions utilizing terms such as “implementing,” “inputting,” “operating,” “deciding,” “detecting,” “notifying,” “aggregating,” “coordinating,” “applying,” “comparing,” “engaging,” “predicting,” “recording,” “analyzing,” “determining,” “identifying,” “classifying,” “generating,” “extracting,” “receiving,” “processing,” “acquiring,” “performing,” “producing,” “providing,” “prioritizing,” “arranging,” “matching,” “measuring,” “storing,” “signaling,” “proposing,” “altering,” “creating,” “computing,” “loading,” “inferring,” or the like, refer to actions and processes of a computing system or similar electronic computing device or processor. The computing system or similar electronic computing device manipulates and transforms data represented as physical (electronic) quantities within the computing system memories, registers or other such information storage, transmission or display devices.

Various embodiments described herein may be discussed in the general context of computer-executable instructions residing on some form of computer-readable storage medium, such as program modules, executed by one or more computers or other devices. By way of example, and not limitation, computer-readable storage media may comprise non-transitory computer storage media and communication media. Generally, program modules include routines, programs, objects, components, data structures, etc., that perform particular tasks or implement particular abstract data types. The functionality of the program modules may be combined or distributed as desired in various embodiments.

Computer storage media includes volatile and nonvolatile, removable and non-removable media implemented in any method or technology for storage of information such as computer-readable instructions, data structures, program modules or other data. Computer storage media includes, but is not limited to, random access memory (RAM), read only memory (ROM), electrically erasable programmable ROM (EEPROM), flash memory or other memory technology, compact disk ROM (CD-ROM), digital versatile disks (DVDs) or other optical storage, magnetic cassettes, magnetic tape, magnetic disk storage or other magnetic storage devices, or any other medium that can be used to store the desired information and that can be accessed to retrieve that information.

Communication media can embody computer-executable instructions, data structures, and program modules, and includes any information delivery media. By way of example, and not limitation, communication media includes wired media such as a wired network or direct-wired connection, and wireless media such as acoustic, radio frequency (RF), infrared and other wireless media. Combinations of any of the above can also be included within the scope of computer-readable media.

In various embodiments, a personal emergency detection, notification, coordination and response method and system for individuals is disclosed. A monitoring device, for example a digital smart watch, is configured to identify an emergency using built-in sensors. The sensors are operable to perform spectral analysis of skin tissues. Example sensors include, but are not limited to: LEDs (light emitting diodes) and optical detectors for heart rate monitoring, blood perfusion checking, and tissue oxygenation checking; acceleration sensing to sense falls and accidents; and a GPS (Global Positioning Satellite) system for reporting the location of the wearer to interested parties. A communication chip with an associated antenna, and an audio chip can also be included. Deviations in vital signs are used to detect health anomalies. In various embodiments, by aggregating data that is anonymously collected from multiple users, the system constructs models that are compared with data from a specific user, to warn the user before an emergency occurs, for certain classes of health incidents.

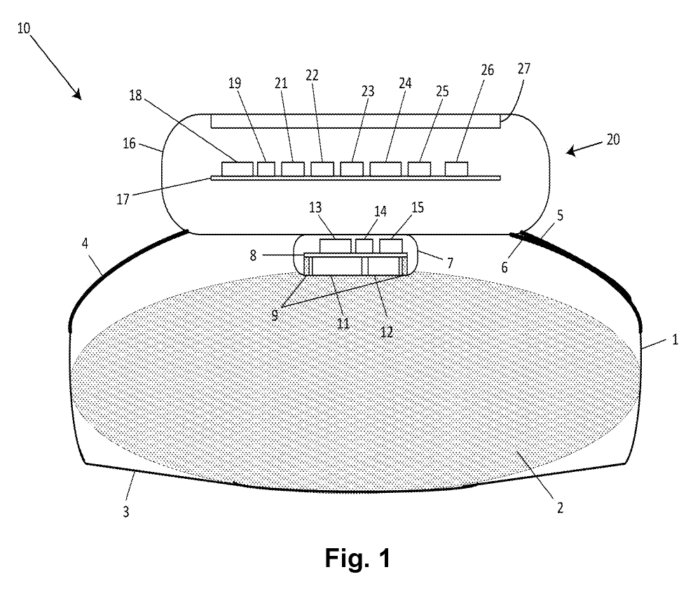

FIG. 1 is a cross-sectional view of an exemplary wrist mounted health monitoring system 10 in accordance with various embodiments of the present disclosure. A clasp or band 1 secures the device 10 to a user's wrist 2, and preferably employs a stiffening element 3 to provide a suitable force for pressing the device against the user's wrist 2. A cellular antenna 4 supports cellular communications. A Global Navigation Satellite System (GNSS) antenna 5 is also provided, used for communications with a Global Navigation Satellite System, a Global Positioning Satellite (GPS) system implemented in the United States, as well as in other nations. A diversity antenna 6 is additionally provided to augment the other antennas and improve their performance. A flex circuit (not shown) embedded in the clasp or band 1 may be used to connect the antennas to corresponding circuits on a second printed circuit board 17, to be described.

A sensor module 7 is shown within FIG. 1, with its bottom surface pressing against the user's skin; this module may be described as a skin tissue sensor. Module 7 includes, but is not limited to, a first printed circuit board 8, an array of conductive elements or capacitors 9 for confirming good contact with the user's wrist 2; an array of sensing circuits (not shown) associated with the array of conductive elements or capacitors for determining proper disposition of the skin tissue sensor relative to the user's body; light emitting diodes (LEDs) 11 having multiple operating frequencies; photo detectors 12 for measuring light originating from diodes 11 that subsequently diffuses through the user's blood and skin tissue; a temperature sensor 13 for recording ambient temperature and supporting calibration of sensor module 7; an accelerometer 14 for sensing falls and accidents, and for confirming user activity; and a first microprocessor 15 for controlling the sensor module 7. Light emitting diodes 11 preferably comprise a plurality of spaced-apart LEDs operating at multiple frequencies. Light emitting diodes 11 and photo detectors 12 are preferably arranged in a predetermined array format, such that absorption spectra may be measured. The absorption spectra may be used to determine vital signs of the user, and the vital signs may be used to make inferences about the user's health.

Within FIG. 1, an enclosure 16 surrounds a computer module 20 comprising, for example and without limitation, the following elements: a second printed circuit board 17; an embedded subscriber identification module (SIM) 18; cellular module 19 for supporting cellular communications; a second microprocessor 21 for controlling computer module 20, flash memory 22; SDRAM (synchronous dynamic random access memory) 23; battery and power management controller 24; charging interface 25 for charging a battery (not shown); a GNSS (Global Navigation Satellite System) module 26; and a touch/display screen 27. Computer module 20 preferably also comprises a voice chip (not shown), for signaling the user and potential local responders.

It is noted that the health monitoring system 10 may not include all of the elements illustrated by FIG. 1. In addition, the health monitoring system 10 can be implemented to include one or more elements not illustrated by FIG. 1. It is pointed out that the health monitoring system 10 can be utilized or implemented in any manner similar to that described and/or shown by the present disclosure, but is not limited to such.

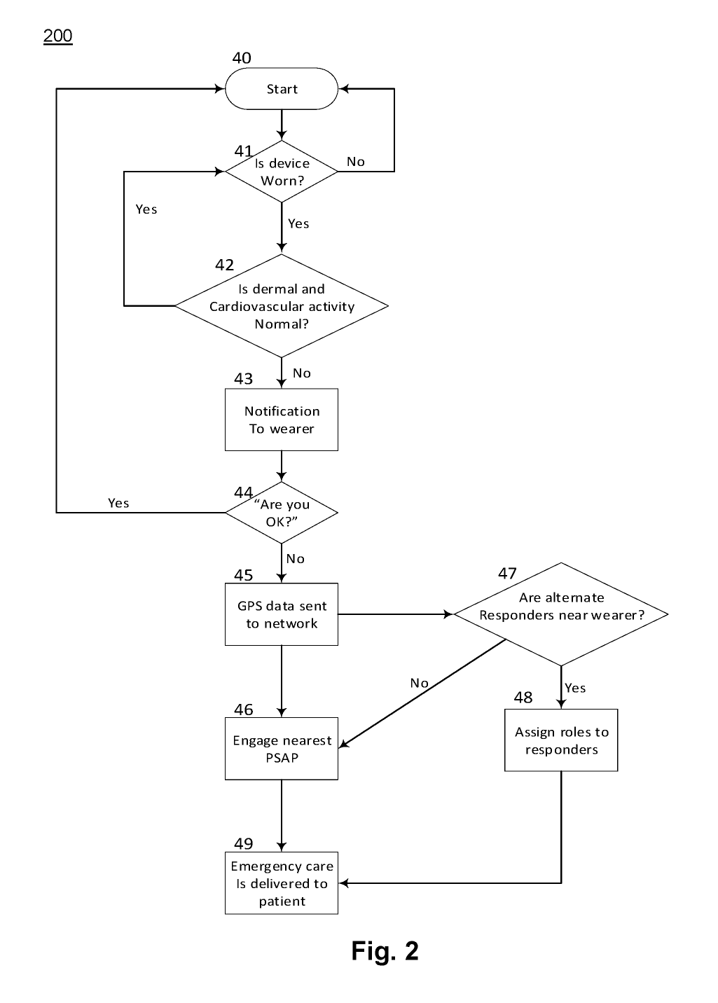

FIG. 2 is a flow chart of a method 200 representing a preferred embodiment in accordance with the present disclosure. Although specific operations are disclosed in FIG. 2, such operations are exemplary. The method 200 may not include all of the operations illustrated by FIG. 2. Also, method 200 may include various other operations and/or variations of the operations shown. Likewise, the sequence of the operations of flow diagram 200 can be modified. It is appreciated that not all of the operations in flow diagram 200 may be performed. In various embodiments, the operations of the flow chart 200 are executed by the second microprocessor 21, according to instructions contained in flash memory 22 or SDRAM 23. Start bubble 40 is entered at power on, or reset of health monitoring system 10. Decision block 41 determines if the device is properly seated against the user's wrist 2, taking advantage of bending forces generated in stiffening element 3. If not, the method 200 proceeds to start bubble 40. If the device is properly worn, system 10 operates sensor module 7 to determine if dermal and cardiovascular activity is normal; this condition may also be described as normal vital signs. Measurements involve using the sensor module to perform spatially resolved spectroscopy, which may be described as Near Infrared Spectroscopy (NIRS). Normal dermal activity may comprise tissue oxygenation measurements, or blood perfusion checking. Normal cardiovascular activity typically comprises heart rate monitoring and detection of anomalies such as fibrillation or unusual cardiac rhythms. Decision block 42 determines if the activity is normal or not.

If the activity measured in decision block 42 is normal, the method 200 proceeds to decision block 41. However, if the activity measured in decision block 42 is not normal, the wearer is notified in block 43. The process 200 flows to decision block 44 wherein the user is asked if he or she is okay. If the user responds in the positive, the method 200 proceeds to start bubble 40. However, if the user responds in the negative, location (GPS or GNSS) data is sent in block 45 to a support network, which typically includes a Public Safety Answering Point (PSAP). An example of a PSAP is a 911 call center, which will be engaged in block 46 by the encoded messages from health monitoring system 10. In addition, other medical resources may also be called upon, as in block 47. The other resources may include medical personnel such as doctors or nurses, or medical equipment such as defibrillators. If assistance is offered by a local responder, then health system 10 will coordinate the emergency response activities and assign roles to the local responders in block 48. If either the PSAP or local responders are available and engaged, emergency care will be delivered to the user as in block 49.

In a preferred embodiment in accordance with the present disclosure, health system 10 will be configurable to aggregate data from multiple users anonymously, and apply additional analysis to establish norms of behavior, and by comparing user data against the norms of behavior, predict some user health emergencies before they would otherwise occur. The additional analysis preferably includes machine learning.

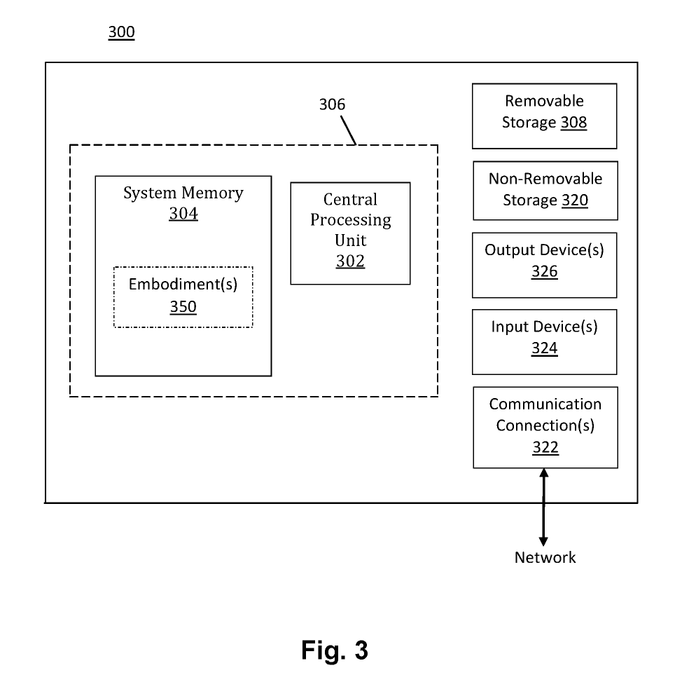

FIG. 3 shows a block diagram of an example of a computing system 300 upon which one or more various embodiments described herein may be implemented in accordance with various embodiments of the present disclosure. In a basic configuration, the system 300 includes at least one processing unit 302 and memory 304. This basic configuration is illustrated in FIG. 3 by dashed line 306. The system 300 may also have additional features and/or functionality. For example, the system 300 may also include additional storage (e.g., removable and/or non-removable) including, but not limited to, magnetic or optical disks or tape. Such additional storage is illustrated in FIG. 3 by removable storage 308 and non-removable storage 320.

The system 300 may also contain communications connection(s) 322 that allow the device to communicate with other devices, e.g., in a networked environment using logical connections to one or more remote computers. Furthermore, the system 300 may also include input device(s) 324 such as, but not limited to, a voice input device, touch input device, keyboard, mouse, pen, touch input display device, etc. In addition, the system 300 may also include output device(s) 326 such as, but not limited to, a display device, speakers, printer, etc.

In the example of FIG. 3, the memory 304 includes computer-readable instructions, data structures, program modules, and the like associated with one or more various embodiments 350 in accordance with the present disclosure. However, the embodiment(s) 352 may instead reside in any one of the computer storage media used by the system 300, or may be distributed over some combination of the computer storage media, or may be distributed over some combination of networked computers, but is not limited to such.

It is noted that the computing system 300 may not include all of the elements illustrated by FIG. 3. Moreover, the computing system 300 can be implemented to include one or more elements not illustrated by FIG. 3. It is pointed out that the computing system 300 can be utilized or implemented in any manner similar to that described and/or shown by the present disclosure, but is not limited to such.

The foregoing descriptions of various specific embodiments in accordance with the present disclosure have been presented for purposes of illustration and description. They are not intended to be exhaustive or to limit the present disclosure to the precise forms disclosed, and many modifications and variations are possible in light of the above teaching. The present disclosure is to be construed according to the Claims and their equivalents.

What is claimed is:

1. A wearable health sensing system comprising:
an enclosure;
a clasp or band for attaching the enclosure to a user's body;
a processor;
a memory containing instructions to be executed by the processor;
a communication chip;
an antenna associated with the communication chip;
a Global Positioning Satellite (GPS) chip;
a skin tissue sensor;
an energy storage device;
a charging interface for replenishing or recharging the energy storage device;
wherein the processor is operable to process data sensed by the skin tissue sensor to detect anomalies that may occur in the user's vital signs, decide on an appropriate response, notify a support system, and coordinate emergency care of the user;
wherein the health sensing system is further configurable to aggregate data from multiple users anonymously, and apply additional analysis to establish norms of behavior, and by comparing user data against the norms of behavior, predict some user health emergencies before they would otherwise occur.

2. The wearable health sensing system as described in claim 1 wherein the skin tissue sensor comprises an array of photo diodes and detectors.

3. The wearable health sensing system as described in claim 1 wherein the skin tissue sensor is operable to perform spectral analysis.

4. The wearable health sensing system as described in claim 1 further comprising an accelerometer for sensing activity level and physical mishaps.

5. The wearable health sensing system as described in claim 1 further comprising a speaker for producing sound, for signaling the user or nearby persons.

6. The wearable health sensing system as described in claim 1 further comprising an interactive display.

7. The wearable health sensing system as described in claim 1 further comprising a capacitive sensor for the wearable health sensing system to determine if good contact is made between the skin tissue sensor and the user's skin.

8. The wearable health sensing system as described in claim 1 wherein the additional analysis comprises machine learning.

9. The wearable health sensing system as described in claim 1 further comprising a diversity antenna to enhance the performance of the antenna.

10. A method for identifying a medical emergency of a user, notifying support systems, and coordinating emergency care, the method comprising:
providing a digital monitoring device in the form of a wearable device;
providing a processor and memory for operating the digital monitoring device;
providing a plurality of spaced apart light emitting diodes (LEDs) having multiple frequencies;
positioning the spaced apart LEDs adjacent the user's skin;
measuring reflections from the user's skin;
recording the measured reflections as absorption spectra;
analyzing the absorption spectra to determine the vital signs;
detecting an abnormality;
deciding on an appropriate response to the abnormality;
notifying one or more support systems if an abnormality has occurred;
coordinating emergency care to be administered to the user;
aggregating data from multiple users to anonymously create standards for normal and abnormal data sets; and
comparing the user's data set against the normal and abnormal data sets to provide early warning to the user of a potential health emergency.

11. The method as described in claim 10 further comprising signaling the user via a vibrating element in the digital monitoring device.

12. The method as described in claim 10 further comprising signaling of the user via tone or voice.

13. The method as described in claim 10 further comprising signaling of nearby persons via tone or voice.

14. The method as described in claim 10 further comprising providing confirmation by the user that an incident has occurred.

15. The method as described in claim 10 further comprising assuming confirmation that an incident has occurred if the user is incapacitated.

16. The method as described in claim 10 further comprising coordinating emergency responses through a dispatch center.

17. The method as described in claim 10 further comprising engaging additional responders who are in the same location as the user.

18. The method as described in claim 10 further comprising providing acceleration sensing and using this data to notify interested parties of falls or accidents.

19. The method as described in claim 10 further comprising notifying interested parties of health incidents detected by the digital monitoring device.

20. The method as described in claim 10 further comprising coordinating individuals and systems based on data to expedite emergency care.

21. The method as described in claim 10 further comprising providing tissue oxygenation sensing and reporting.

22. The method as described in claim 10 further comprising providing pulse rate sensing and reporting.

23. The method as described in claim 10 further comprising determining if the wearable device is properly positioned adjacent the user's skin.

24. The method as described in claim 10 further comprising providing ambient temperature sensing and using this information to calibrate the digital monitoring device.

25. The method as described in claim 10 wherein the LED measurements utilize spatially resolved spectroscopy.

26. A health sensing system comprising:
a processor and memory for operating the health sensing system;
a plurality of spaced apart LEDs in contact with the user's body and having multiple frequencies;
a plurality of spaced apart photo detectors for measuring light sourced from the spaced apart LEDs that subsequently diffuses through the user's body;
an array of conductive components in contact with the user's body;
an array of sensing circuits associated with the array of conductive components for determining correct disposition of the health sensing system relative to the user's body;
absorption spectra determined from the measurements of light sourced from the spaced apart LEDs that subsequently diffuses through the user's body;
user's vital signs determined from the absorption spectra; and
the processor operable to make inferences about the health of the user determined from the vital signs;
the processor operable to aggregate data from multiple users to anonymously create standards for normal and abnormal data sets; and
the processor operable to compare the user's vital signs against the normal and abnormal data sets to provide early warning to the user of a potential health emergency.
